(19) United States
(12) Patent Application Publication
(10) Pub. No.: US 2003/0144259 A1
(43) Pub. Date: Jul. 31, 2003

(54) Breast cancer hormonal therapy

(76) Inventors: Giorgio Massimini, Abbiategrasso (IT); Robert Paridaens, Bruxelles (BE); Jean-Pierre Lobelle, Beernem (BE); Gabriella Piscitelli, Milano (IT)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/204,802

(22) PCT Filed: Feb. 20, 2001
(86) PCT No.: PCT/EP01/01883

(30) Foreign Application Priority Data
Mar. 3, 2000 (GB) 0005257.1

(51) Int. Cl. A61K 31/57

(52) U.S. Cl. 514/177

(57) ABSTRACT

Exemestane is disclosed for use in the first-line treatment of metastatic, advanced hormone-dependent breast cancer, particularly in post-menopausal woman.

The present invention is in the field of endocrine therapy in metastatic, advanced breast cancer.

Breast cancer is the most common cancer diagnosis and the second leading cancer-related cause of death in American women. At diagnosis, approximately 5% of women have metastatic disease.

Endocrine therapy is generally well tolerated and because of the favourable therapeutic index, it is the treatment of first choice for women with metastatic breast cancer. In fact, randomized trials show no adverse effect on survival for patients initially treated with endocrine therapy rather than chemotherapy. As yet, the standard, first-line endocrine agent is tamoxifen. Second-line therapy agents which include progestins and aromatase inhibitors as yet are known to have in fact more side effects than tamoxifen.

Well documented adverse effects of tamoxifen concerns mainly the reproductive organs, the most worrying being its carcinogenity for the endometrium. Cases of ocular toxicity have been also reported. These cases involved internal crystalline deposits, impaired visual acuity, macular oedema, keratopathy and optic neuritis. Others side effects include, for instance, hot flashes, anorexia, nausea, vomiting and skin rush. Clinical and laboratory data suggest that tamoxifen reduces the cytolytic effect of melphalan, cyclophosphamide and 5-fluorouracyl.

Moreover, in approximately 5% of patients with skin or bone matastases, tamoxifen causes a tumor “flare” manifested by an increase in size, number, and discomfort of skin lesions and by increasing bone pain and/or hypercalcemia. Such reactions generally occur within days or weeks of treatment initiation. Flare reactions may occur in association with other hormonal therapies such as estrogens, androgens, progestins, and ablative therapies.

On the other hand, for instance aromatase inhibitor amninoglutethimide may even yield a response rate similar to tamoxifen when used as a first-line therapy in metastatic breast cancer. Unfortunately side effects of aminoglutethimide are considerable, they occur in about 35% of patients and require discontinuation of drug in about 5%. The major toxicities are lethargy (36%), a transient maculopapular rush (25%), dizziness (15%) and nausea and vomiting (10%), with severe myelosuppression reported in less than 1% of patients. Severity and frequence of these side effects have thus make arninoglutetimide less desirable than tamoxifen as first-line endrocrine treatment agent.

The inventors of the present invention have surprisingly found that aromatase inhibitor exemestane is both more active and better tolerated than tamoxifen as first-line endocrine agent in metastatic, advanced breast cancer.

Accordingly a first object of the present invention is to provide a method for the first-line treatment of metastatic, advanced hormone-dependent breast cancer comprising administering a patient, in need of such first-line treatment, a therapeutically effective amount of exemestane or a pharmaceutical composition containing it.

A further object of the invention is to provide the use of exemestane in the preparation of a pharmaceutical composition for use in first-line treatment of metastatic, advanced hormone-dependent breast cancer, in particular in a post-menopausal woman.

Aromatase inhibitor exemestane is a well-known compound, it is for instance disclosed by U.S. Pat. No. 4,808,616. U.S. Pat. No. 4,808,616 teaches the use of exemestane in the treatment of advanced hormone-dependent breast cancer. However this is the first time that exemestane is specifically described as a first-line endocrine agent for treating metastatic, advanced breast cancer.

As known, a first-line endocrine agent is the first choice endocrine agent for treating a patient who has never been treated before with endocrine agents (except in the case of possible adjuvant therapy after surgery), whereas e.g. a third line endocrine agent is an endocrine agent which is administered to a patient previously treated with at least two hormonal agents. From the above it will be appreciated that the conditions of a first-line treated patient suffering from metastatic, advanced breast cancer and a second- (or third-) line treated patient suffering from advanced breast cancer are quite different, as for example the hormone-receptor status and extent of disease.

The valuable properties of exemestane as a first-line endocrine agent in metastatic, advanced breast cancer are shown for instance by the following randomized phase II trial aimed at examining activity and safety of exemestane (E) at the dosage of 25 mg/day per os versus tamoxifen (T) at the dosage of 20 mg/day per os in a first-line treatment of metastatic, advanced breast cancer (MBC), in postmenopausal women. According to the trial framework, of 97 randomized patients, 63 (31 E and 32 T) and 76 (37E, 39 T) were evaluable for response and toxicity, respectively. Patient characteristics were week balanced. Six and 8 patients in the E and T arms, respectively, had received adjuvant T.

Results: The most frequent grade 2/3 adverse events were fatigue (E 54%, T 12.8%), pain (E 10.8%, T 17.9%), hot flushes E 2.7%, T 15.4%), sweating (E 0, T 10.3%), edema (E 2.7%, T 7.7%), infection (E 5.4%, T 5.1%), nausea (E 2.7%, T 7.7%), dyspnea (E 10.8%, T 7.7%) and weight gain (E 5.4%, T 5.1%).

Exemestane was found to be significantly more active than tamoxifen in MBC, as shown in the Table herebelow.

| | Exemestane | Tamoxifen |
|---|---|---|
| | n = 31 | n = 32 |
| Median time to progression, months | 8.9 | 5.2 |
| CR, % | 9.7 | 3.1 |
| CR + PR(OR), % | 42 | 16 |
| CR + PR + NC ≧ 6 months, % | 58 | 31 |

In the table CR means Complete Remission, PR means Partial Remission, NC means No Change and OR means Objective Response.

In view or the above comparative clinical results, we can safely state that exemestane is both more active and better tolerated than the standard, first-line endocrine agent tamoxifen.

Exemestane, at the present time, is thus a safer tool as a first-line endocrine agent in the treatment of metastatic, advanced breast cancer.

From the pharmacological point of view, the valuable biological properties of exemestane may be found in its peculiar mechanism of aromatase inactivation. The aromatase enzyme (450ₐᵣₒₘ) is a specific form of cytochrome P450 hemoprotein composed of a P450 (heme) moiety and a peptidic moiety. The enzyme catalyzes a multistep reaction leading to aromatization of the A ring of the androgen substrate (mainly androstenedione) to estrone, requiring the presence of the cofactor NADPH. After this enzymatic reaction, the enzyme molecule is once more available to perform a new aromatization.

The exemestane's mechanism of aromatase inhibition has been extensively studied and the compound has been found to cause enzyme inactivation. In fact exemestane, structurally related to the natural substrate androstenedione, is initially recognized by the aromatase enzyme as a false substrate, therefore competes with androstenedione at the active site of the enzyme. The compound is then transformed (through and NADPH-dependent mechanism) to an intermediate which binds irreversibly to the enzyme causing its inactivation (also known as suicide inhibition). Therefore the enzyme is definitely inactivated and de novo enzyme synthesis is required for oestrogen production.

As used herein, the term “therapeutically effective (antineoplastic) amount ” refers to an amount which is effective, upon single or multiple dose administration to the patient, in controlling the growth of the neoplasm or in prolonging the survival of the patient beyond that expected in the absence of such treatment. As used herein, “controlling the growth ” of the neoplasm refers to slowing, interrupting, arresting or stopping its growth and it does not necessarily indicates a total elimination of the neoplasm.

The dosage of exemestane to be used is, of course dependent on various factors such as the human to be treated (e.g. male or late premenopausal or postmenopausal female, age, weight and general status of health), the severity of the symptoms, the disorder to the accompanying treatment with other pharmaceuticals, or the frequency of the treatment.

Exemestane can for example be administered to a postmenopausal woman orally in a dosage range varying from about 5 to about 50 mg/day, preferably, from about 10 to about 25 mg/day, and in particular at about 25 mg/day, or parenterally from about 50 to about 500 mg, in particular from about 100 to about 250 mg.

In effecting treatment of a patient afflicted with an metastatic, advanced breast cancer exemestane can be administered in any form or mode which makes the compound bioavailable in effective amounts, including oral and parenteral routes. For example, it can be administered orally, subcutaneously, intraperitoneally, intramuscularly, intravenously, transdermally, and the like. Oral or intramuscular administration is generally preferred. One skilled in the art of preparing formulations can readily select the proper form and mode of administration depending upon the particular circumstances, including the stage of the disease.

For example, U.S. Pat. No. 4,808,616 discloses the preparation of pharmaceutical compositions comprising exemestane and a suitable carrier or excipient.

1. Use of exemestane in the preparation of a pharmaceutical composition for use in first-line treatment of metastatic, advanced hormone-dependent breast cancer.

2. Use, according to claim 1, for use in first-line treatment of metastatic, advanced hormone-dependent breast cancer in a post-menopausal woman.

3. Use, according to claim 1 or 2, wherein the pharmaceutical composition is suitable for oral administration and the amount of exemestane is from about 5 to about 50 mg/day.

4. Use, according to claim 3, wherein the amount of exemestane is from about 10 to about 25 mg/day.

5. Use, according to claim 3, wherein the amount of exemestane is about 25 mg/day.

6. Use, according to claims 1 or 2, wherein the pharmaceutical composition is for parenteral administration and the amount of exemestane is from about 50 to about 500 mg.

7. Use, according to claims 1 or 2, wherein the amount of exemestane is from about 100 to about 250 mg.

8. A method for the first-line treatment of metastatic, advanced hormone-dependent breast cancer comprising administering to a patient, in need of such first-line treatment, a therapeutically effective amount of exemestane or a pharmaceutical composition containing it.

9. A method for the first-line treatment of metastatic, advanced hormone-dependent breast cancer comprising orally administering to a patient, in need of such first-line treatment, a therapeutically effective amount of exemestane ranging from about 5 to about 50 mg/day or a pharmaceutical composition containing it.

10. A method according to claim 9, wherein the amount of exemestane is from about 10 to about 25 mg/day.

11. A method according to claim 9, wherein the amount of exemestane is about 25 mg/day.

12. A method for the first-line treatment of metastatic, advanced hormone-dependent breast cancer comprising administering to a postmenopausal woman, in need of such first-line treatment, a therapeutically effective amount of exemestane or a pharmaceutical composition containing it.

13. A method for the first-line treatment of metastatic, advanced hormone-dependent breast cancer comprising orally administering to a postmenopausal woman, in need of such first-line treatment, a therapeutically effective amount of exemestane ranging from about 5 to about 50 mg/day or a pharmaceutical composition containing it.

14. A method according to claim 13, wherein the amount of exemestane is from about 10 to about 25 mg/day.

15. A method according to claim 13, wherein the amount of exemestane is about 25 mg/day.

16. A method for the first-line treatment of metastatic, advanced hormone-dependent breast cancer comprising parenterally administering to a postmenopausal woman, in need of such first-line treatment, a therapeutically effective amount of exemestane ranging from about 50 to about 500 mg or a pharmaceutical composition containing it.

17. A method according to claim 16, wherein the amount of exemestane is from about 100 to about 250 mg.
